(19) United States
(12) Patent Application Publication
(10) Pub. No.: US 2003/0181418 A1
(43) Pub. Date: Sep. 25, 2003

(54) Pharmaceutical composition

(75) Inventors: Seiichiro Kumakura, Saitama-shi (JP); Tomoko Nakajima, Tokyo (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: SANKYO COMPANY, LIMITED, Tokyo (JP)

(21) Appl. No.: 10/377,230

(22) Filed: Feb. 28, 2003

(30) Foreign Application Priority Data
Mar. 1, 2002 (JP) 2002-055356

(51) Int. Cl. A61K 31/715

(52) U.S. Cl. 514/59

(57) ABSTRACT

A pharmaceutical composition which contains (i) a complex of (a) one or more OCIF substances selected from the group consisting of an osteoclastogenesis inhibitory factor, an analog thereof and a variant thereof, and (b) a polysaccharide substance comprising a polysaccharide or a derivative thereof and (ii) a substance suppressing production of a prostaglandin and/or a substance competing an action of a prostaglandin. The invention also provides a method for treatment or prevention of a bone metabolic disease by administering to a patient in need thereof, an effective amount of (i) a complex of (a) one or more OCIF substances selected from the group consisting of an osteoclastogenesis inhibitory factor, an analog thereof and a variant thereof, and (b) a polysaccharide substance comprising a polysaccharide or a derivative thereof, and of (ii) a substance suppressing production of prostaglandin and/or a substance competing an action of prostaglandin.

This application claims the benefit of the filing date of JP 2002-055356 filed Mar. 1, 2002 which is hereby incorporated herein in its entirety by this reference.

### TECHNICAL FIELD OF THE INVENTION

The present invention relates to a pharmaceutical composition which contains a complex of one or more of substances selected from the group consisting of an osteoclastogenesis inhibitory factor (hereinafter referred to as “OCIF”, see, WO96/26217), an analog thereof and a variant thereof, and a polysaccharide or a derivative thereof (hereinafter referred to as “polysaccharide substance”) wherein the OCIF substance and the polysaccharide substance are bound in a molecular ratio of 1:1 to 1:8 and a substance suppressing production of prostaglandin and/or a substance competing an action of prostaglandin; as well as an agent for prevention or treatment of bone metabolic diseases containing as an active ingredient a substance suppressing production of prostaglandin and/or a substance competing an action of prostaglandin; or the like.

### DESCRIPTION OF THE PRIOR ART

Bones occupy about 99% of calcium in a living body, and play an important role in maintaining homeostasis of calcium concentration in blood by bone formation and bone resorption. If osteoclasts playing a main role in a process of bone resorption are formed or activated abnormally, bone resorption will be promoted, calcium concentration in blood will increase, and hypercalcemia will be caused. It is known that cytokines will be secreted abnormally in the case that cancer transfers to a bone, and hypercalcemia will be caused. Also in that case, promotion of bone resorption by osteoclasts causes increase of calcium concentration in blood. Prognosis of a cancer patient accompanied by cancerous hypercalcemia is generally poor.

Moreover, in rheumatism and osteoarthritis, such as rheumatoid arthritis or the like, abnormal formation or abnormal activation of osteoclasts is known as one of main causes of various symptoms shown in bones and joints. Since rheumatism and osteoarthritis, such as rheumatoid arthritis, are accompanied by an intense ache, they are remarkably disadvantageous to a life of a patient.

Furthermore, when the balance of bone resorption and bone formation continuously inclines toward bone resorption side due to lowering of secretion of female sex hormones after menopause or aging, a bone density is lowered and symptoms of osteoporosis are shown. Osteoclasts take charge of bone resorption also in this case. When an old age patient with a high risk of osteoporosis suffers a fracture, a possibility of becoming bedridden is high. Therefore, it is also a social subject.

Conventionally, there have been used for these diseases, therapy of supplementing hormones such as estrogen, therapeutic agents for suppressing activity of osteoclasts such as bisphosphonates or calcitonins. However, hormones may have side reactions such as raising risk of oncogenesis, induction of endometriosis, abnormal bleeding from genitals, or the like. Although it is known that bisphosphonates easily bind to calcium and thus accumulated at a bone, some researchers have doubt that strength of bone is improved to what extent, and a danger of kidney hindrance is also reported. Increase of a bone density of calcitonins is transient, and there have been pointed out that there is a rebound due to interruption of administration, lowering of the effect of a medicine due to appearance of immunogenicity or antigenicity and antibody production in human body in the case of calcitonin originating in animals other than human, and the like. Although osteoclasts take charge of bone resorption which is a factor of increase of calcium concentration in blood, the above-mentioned existing medicines have no activity of suppressing formation of the osteoclast. Therefore, they are not considered to be enough medicines for fundamental treatment of hypercalcemia and bone metabolic diseases.

OCIF is known as an endogenic protein suppressing differentiation of an osteoclast precursor cell to an osteoclast, and bone resorption activity of the mature osteoclast (see, WO96/26217 or the like). It was expected that the above-mentioned diseases resulting from bone resorption can be treated fundamentally, if formation of the osteoclast mainly takes charge of bone resorption itself and the bone resorption activity of the mature osteoclast can be suppressed by OCIF. However, OCIF is a basic protein of which an isoelectric point is near 9, and disappears promptly out of circulation blood.

It is indicated in WO00/24416 that the retentivity in blood of OCIF is improved and the effect as a medicine is improved by mixing OCIF and a polysaccharide or a derivative thereof.

The substance suppressing production of prostaglandin and the substance competing an action of prostaglandin are used mainly as anti-inflammatory and/or analgesic agent in treatment of rheumatism such as rheumatoid arthritis or the like, or osteoarthritis, and is not applied to fundamental prevention or fundamental treatment of bone metabolic diseases.

### OBJECT OF THE INVENTION

A complex of the OCIF and dextran sulfate which is a derivative of polysaccharide, obtained by treating the OCIF and dextran sulfate which is a derivative of polysaccharide under an alkaline condition has a significantly improved retentivity in blood as compared with a conventional known composition of OCIF and dextran sulfate. The inventors have found that the pharmaceutical effect can be reinforced by combined use of the composition and a substance suppressing production of prostaglandin and/or a substance competing an action of prostaglandin, and thereby have completed the present invention.

### MEANS FOR SOLVING THE PROBLEM

The present invention relates to the following pharmaceutical compositions:

(1) A pharmaceutical composition which contains [i] a complex of one or more of substances selected from the group consisting of an osteoclastogenesis inhibitory factor (hereinafter referred to as “OCIF”), an analog thereof and a variant thereof (hereinafter referred to as “the OCIF substance”), and a polysaccharide or a derivative thereof (hereinafter referred to as “the polysaccharide substance”), wherein the OCIF substance and the polysaccharide substance are bound in a molecular ratio of 1:1 to 1:8; and

[ii] a substance suppressing production of prostaglandin and/or a substance competing an action of prostaglandin.

(2) The pharmaceutical composition according to (1) wherein the polysaccharide or a derivative thereof is dextran sulfate.

(3) The pharmaceutical composition according to (2) wherein an average molecular weight of the dextran sulfate is 1800 to 6000.

(4) The pharmaceutical composition according to any one of (1) to (3) wherein the substance suppressing production of prostaglandin and/or the substance competing an action of prostaglandin is non-steroidal anti-inflammatory drug.

(5) The pharmaceutical composition according to (4) wherein the non-steroidal anti-inflammatory drug is cyclooxygenase inhibitor.

(6) The pharmaceutical composition according to (5) wherein the cyclooxygenase inhibitor is loxoprofen sodium or celecoxib.

(7) The pharmaceutical composition according to any one of (1) to (6) wherein the complex of the OCIF substance and the polysaccharide substance is produced by the following steps [i] and [ii]:

[i] a step of mixing the OCIF substance and the polysaccharide substance and keeping a temperature of them;

[ii] a step of removing a free polysaccharide or a derivative thereof obtained in.

(8) The pharmaceutical composition wherein a temperature of the OCIF substance and the polysaccharide substance is kept under alkaline condition in the step [i] according to (7).

(9) The pharmaceutical composition according to (8) wherein a temperature of the OCIF substance and the polysaccharide substance is kept under the condition of pH 10 to 11 in the step [i] according to (7).

(10) The pharmaceutical composition according to any one of (7) to (9) wherein the free polysaccharide or a derivative thereof is removed by gel filtration.

(11) An agent for prevention or treatment of bone metabolic diseases which contains as an active ingredient:

[i] a complex of one or more of substances selected from the group consisting of an osteoclastogenesis inhibitory factor (hereinafter referred to as “OCIF”), an analog thereof and a variant thereof (hereinafter referred to as “the OCIF substance”), and a polysaccharide or a derivative thereof (hereinafter referred to as “the polysaccharide substance”), wherein the OCIF substance and the polysaccharide substance are bound in a molecular ratio of 1:1 to 1:8; and

[ii] a substance suppressing production of prostaglandin and/or a substance competing an action of prostaglandin.

(12) The agent for prevention or treatment of bone metabolic diseases according to (11) wherein the polysaccharide or a derivative thereof is dextran sulfate.

(13) The agent for prevention or treatment of bone metabolic diseases according to (12) wherein an average molecular weight of the dextran sulfate is 1800 to 6000.

(14) The agent for prevention or treatment of bone metabolic diseases according to any one of (11) to (13) wherein the substance suppressing production of prostaglandin and/or the substance competing an action of prostaglandin is non-steroidal anti-inflammatory drug.

(15) The agent for prevention or treatment of bone metabolic diseases according to (14) wherein the non-steroidal anti-inflammatory drug is cyclooxygenase inhibitor.

(16) The agent for prevention or treatment of bone metabolic diseases according to (15) wherein the cyclooxygenase inhibitor is loxoprofen sodium or celecoxib.

(17) The agent for prevention or treatment of bone metabolic diseases according to any one of (11) to (16) wherein the complex of the OCIF substance and the polysaccharide substance is produced by the following steps [i] and [ii]:

[i] a step of mixing the OCIF substance and the polysaccharide substance and keeping a temperature of them;

[ii] a step of removing a free polysaccharide or a derivative thereof obtained in.

(18) The agent for prevention or treatment of bone metabolic diseases wherein a temperature of the OCIF substance and the polysaccharide substance is kept under alkaline condition in the step [i] according to (17).

(19) The agent for prevention or treatment of bone metabolic diseases according to (18) wherein a temperature of the OCIF substance and the polysaccharide substance is kept under the condition of pH 10 to 11 in the step [i] according to (17).

(20) The agent for prevention or treatment of bone metabolic diseases according to any one of (17) to (19) wherein the free polysaccharide or a derivative thereof is removed by gel filtration.

### EMBODIMENTS OF THE INVENTION

An osteoclastogenesis inhibitory factor (hereinafter referred to as “OCIF”), an analog thereof and a variant thereof of the present invention can be obtained by a method described in WO96/26217 or WO 97/23614, as a natural protein extracted and purified from an organ of an animal, body fluid of an animal, or a culture medium of animal cells, or as a recombinant protein produced by host cells such as animal cells and Escherichia coli transformed with a vector containing polynucleotide encoding OCIF, its analog, or its variant.

The OCIF, an analog thereof and a variant thereof of the present invention is preferably deriving from mammalians, such as a human, a rat, a mouse; a rabbit, a dog, a cat, a cow, a swine, a sheep, and a goat, or birds, such as a fowl, a goose, and a turkey, more preferably it is human OCIF, without being limited thereto.

Moreover, OCIF and its analog of the present invention are produced as a polypeptide of a single strand, the molecular weight by SDS-PAGE under a non-reducing condition is about 60000 or about 120000 (see WO96/26217), and it is preferably a dimer of molecular weight about 120000.

In the present invention, an OCIF analog is a protein encoded by a polynucleotide which can be obtained from cDNA library deriving from animal cells, body fluid, or organs by a hybridization method using human OCIF cDNA as a probe. Examples of such OCIF analog include: OCIF2, OCIF3, OCIF4, OCIF5 (see WO96/26217), OCIF originated form animal other than human or the like. Such an OCIF analog can be obtained by obtaining cDNA which encodes an OCIF analog by a hybridization method using human OCIF cDNA as a probe, inserting the it into an expression vector, transforming a host cell therewith, and expressing it by this vector (see WO96/26217).

In the present invention, an OCIF variant means a protein which has an amino acid sequence wherein one or more amino acids are substituted, deleted, added or inserted in human OCIF, has an OCIF activity. Such an OCIF variant can be obtained by substituting, deleting, adding and/or inserting one or more nucleotides in a nucleotide encoding OCIF or an analog thereof according to PCR method, a genetic recombination method or a digesting method using exonuclease or endonuclease such as a restriction enzyme, then transforming a host cell such as an animal cell or Escherichia coli with an expression vector wherein the obtained nucleotide variant is inserted, and purifying the protein from the protein fraction expressed by the host cell according to a conventional method.

Moreover, there is known a molecule which is a short-chain type OCIF wherein a considerable part was deleted from a carboxy terminal of an amino acid sequence of human OCIF and also has an original biological activity (see WO96/26217 and WO97/23614). The short-chain type OCIF having an OCIF activity is also included in the OCIF variant of the present invention. Methionine can be added to such an amino terminus of short-chain type OCIF if needed.

Furthermore, OCIF having an activity as a fusion protein with Fc domain of an immunoglobulin or the like is known (see WO97/23614), and such a fusion protein of OCIF is also included in the OCIF variant of the present invention (see WO97/23614).

Moreover, it is known that OCIF chemically modified with an aqueous polymer or the like has an improved activity. Such a chemically modified OCIF is also included in the OCIF variant of the present invention. OCIF to which polyethylene glycol is bound may exemplify the chemical modified OCIF (see WO97/23614).

Examples of such an OCIF variant of the present invention may include: OCIF-C19S, OCIF-C20S, OCIF-C21S, OCIF-C22S, OCIF-C23S, OCIF-DCR1, OCIF-DCR2, OCIF-DCR3, OCIF-DCR4, OCIF-DDD1, OCIF-DDD2, OCIF-CL, OCIF-CC, OCIF-CDD2, OCIF-CDD1, OCIF-CCR4, OCIF-CCR3, OCIF-CBst, OCIF-CSph, OCIF-CBsp, OCIF-CPst (see WO96/26217), muOPG[22-401]-Fc, muOPG[22-194]-Fc, muOPG[22-185]-Fc, muOPG[22-180]-Fc, muOPG[22-401], muOPG[22-401]C195, muOPG[22-401]C202, muOPG[22-401]C277, muOPG[22-401]C319, muOPG[22-401]C400, muOPG[22-185], muOPG[22-194], muOPG[22-200], muOPG[22-212], muOPG[22-293], muOPG[22-355], huOPG[22-401]-Fc, huOPG[22-201]-Fc, huOPG[22-401]-Fc P26A, huOPG[22-401]-Fc Y28F, huOPG[22-401], huOPG[27-401]-Fc, huOPG[29-401]-Fc, huOPG[32-401]-Fc, MuOPG met[22-194], MUOPG met[22-194] 5k PEG, MuOPG met[22-194] 20k PEG, HuOPG met[22-194]P25A, HuOPG met[22-194]P25A 5k PEG, HuOPG met[22-194]P25A 20k PEG, HuOPG met[22-194]P25A 31k PEG, HuOPG met[22-194]P25A 57k PEG, HUOPG met[22-194]P25A 12k PEG, HuOPG met[22-194]P25A 20k branched PEG, HuOPG met[22-194]P25A 8k PEG dimer, HUOPG met[22-194] P25A disulfide crosslinked (see WO97/23614) or the like. Preferable examples include: OCIF-C19S, OCIF-C20S, OCIF-C21S, OCIF-C22S, OCIF-C23S, OCIF-DCR1, OCIF-DCR2, OCIF-DCR3, OCIF-DCR4, OCIF-DDD1, OCIF-DDD2, OCIF-CL, OCIF-CC, OCIF-CDD2, OCIF-CDD1, OCIF-CCR4, OCIF-CCR3, OCIF-CBst, OCIF-CSph, OCIF-CBsp, OCIF-CPst, muOPG[22-401]-Fc, muOPG[22-194]-Fc, muOPG[22-185]-Fc, muOPG[22-180]-Fc, muOPG[22-401]C195, muOPG[22-401]C202, muOPG[22-401]C319, muOPG[22-401]C400, uOPG[22-194], muOPG[?2-200], muOPG[22-212], muOPG[22-293], muOPG[22-355], huOPG[22-401]-Fc, huOPG[22-201]-Fc, huOPG[22-401]-Fc P26A, huOPG[22-401]-Fc Y28F, huOPG[22-401], huOPG[27-401]-Fc, huOPG[29-401]-Fc, huOPG[32-401]-Fc, MuOPG met[22-194]5k PEG, MuOPG met[22-194]20k PEG, HUOPG met[22-194]P25A 5k PEG, HuOPG met[22-194]P25A 20k PEG, HUOPG met[22-194]P25A 31k PEG, HUOPG met[22-194]P25A 57k PEG, HUOPG met[22-194]P25A 12k PEG, HuOPG met[22-194]P25A 20k branched PEG, HUOPG met[22-194]P25A 8k PEG dimer and a HUOPG met[22-194]P25A disulfide crosslinked.

The OCIF analogues and OCIF variants of the present invention such as described above preferably has a physiological activity which is equivalent to that of OCIF or higher than it, or is almost the same as that of OCIF.

A sugar chain may be added to the OCIF of the present invention, an analog thereof and a variant thereof. Examples of the OCTF, an analog thereof and a variant thereof to which a sugar chain is added may include: recombinant OCIFs produced by animal cells, natural OCIFs isolated from the animal tissue, or the like. Examples of the animal cells preferable for production of the OCIF, an analog thereof and a variant thereof to which a sugar chain is added may include: mammalian cells, such as a Chinese-hamster ovary (hereinafter referred to as “CHO”) or the like. Examples of cells preferable for production of a recombinant OCIF to which a sugar chain is not added may include prokaryotic cells, such as Escherichia coli or the like.

In the present invention, a polysaccharide or derivative thereof is a polymer produced by glycosidic linkage of monosaccharides (glucan) or derivative thereof, and is preferably heteropolysaccharide (heteroglycan) consisting of two or more kinds of monosaccharide, or a derivative thereof. Examples of natural polysaccharide or a derivative thereof may include: hyaluronic acid, chondroitin sulfuric acid, dermatan acid, heparan acid, keratan acid, carrageenan, pectin, heparin or the like. Examples of synthesized polysaccharide of a derivative thereof may include dextran sulfate or the like, preferably dextran sulfate.

In the present invention, dextran sulfate means dextran sulfate or a salt thereof, and is preferably dextran sodium sulfate. Examples of dextran sodium sulfate include: dextran sodium sulfate sulfur 5 (manufactured by Meito Industry), dextran sodium sulfate 5000 and dextran sulfate 10000 (both of them are manufactured by Wako Pure Chemical Industries, Ltd.) and the like.

A molecular weight of dextran sulfate is calculated as follows.

1) Measurement of a Molecular Weight of Dextran

It can be calculated from limiting viscosity (a measuring method of limiting viscosity is described in Japanese pharmacopoeia explanatory, the thirteenth revision, published by Hirokawashoten (1998), a paragraph of dextran 40) by the formula of Sato shown below (see, Japanese pharmacopoeia explanatory, the thirteenth revision, published by Hirokawashoten (1998), a paragraph of dextran 40). Limiting viscosity=9.00×10⁻⁴×molecular weight^{0.50}

2) Measurement of Sulfur Content

Sulfur content on dextran sulfate was measured as weight % by a method described in a paragraph of dextran sulfate sulfur 5 in Japanese pharmacopoeia (14th revision, published by Jihou (2001)).

Although a molecular weight of glucose which is a constituting unit of dextran is originally 180, a substantial molecular weight of glucose in a dextran molecule is a value wherein a molecular weight of water is deducted from 180, namely 162, since glucoses are bound each other by α-1,6 bond in a dextran molecule.

In dextran sulfate, since a hydrogen atom in glucose in the above dextran molecules is replaced by SO₃Na (one gram equivalent 103), the sulfur substitution degree (hereinafter referred to as “substitution degree”) is represented by the following formula.

Sulfur content (% by weight) (32×substitution degree÷(162+102×substitution degree))×100

3) Calculation of a Molecular Weight of Dextran Sulfate

Since a substantial molecular weight of glucose which is a constituting unit of dextran is 162 as described above, a molecular weight of dextran sulfate can be calculated with the following formula.

Molecular weight=molecular weight×(162+102×substitution degree)+162

It is known that dextran sulfate has a certain molecular weight distribution. A molecular weight of dextran sulfate is represented by an average molecular weight thereof in the present invention.

A substitution degree of sulfur (average±standard deviation) of dextran sodium sulfate sulfur 5 (manufactured by Meito Industry) suitably used in the present invention calculated according to the above item 2) is 0.32±0.01 (n=7), and a molecular weight (average±standard deviation) calculated according to the above items 1) to 3) is 1950±70 (n=7). A molecular weight of the dextran sodium sulfate 5000 (manufactured by Wake Pure Chemical Industries, Ltd.) which is also a preferable dextran sulfate is 5000.

Although an average molecular weight of the polysaccharide or a derivative thereof used in the present invention is not limited, the range of an average molecular weight of dextran sulfate which is a preferable polysaccharide or a derivative thereof is generally 1500 to 12000, and is preferably 1800 to 6000.

Although the polysaccharide of the present invention or a derivative thereof may be used as it is, it can be purified or fractionated before use.

A molecular ratio of one or more of substances selected from the group consisting of OCIF, an analog thereof and a variation thereof (the OCIF substance), and a polysaccharide or a derivative thereof (the polysaccharide substance) in the complex of them (the complex of the OCIF substance and the polysaccharide) provided by the present invention is as follows: the OCIF substances: the polysaccharide substance=1:1 to 1:10, preferably 1:1 to 1:8, more preferably 1:1 to 1:6, further preferably 1:1 or 1:4.

It is known that the OCIF will form a dimer as described above (see WO96/26217). Although the OCIF substance can be a homo- or hetero-dimer, or a homo- or hetero-polymer consisting of three or more monomers. A molecular ratio in the present invention is calculated as a number of molecule of the polysaccharide per a monomer of the OCIF substances.

The complex of the OCIF substance and the polysaccharide substance of the present invention can be obtained by keeping them in an aqueous solution, or the like at a temperature in the range specified hereinafter. When keeping them at the specified temperature in an aqueous solution, a range of the concentration of the OCIF substance in the aqueous solution is that wherein a maximum is 10 to 50 mg/ml and the minimum is 0.1 to 5 mg/ml. Preferable range thereof is 1 to 20 mg/ml and the more preferable range is 5 to 10 mg/ml. A range of concentration of the polysaccharide substance in the aqueous solution is that wherein a maximum is 200 mg/ml to 1000 mg/ml, and the minimum is 0.1 to 100 mg/ml. Preferable range thereof is 10 to 500 mg/ml and the more preferable range is 100 to 200 mg/ml. In the aqueous solution, a maximum pH is 11 to 12, and the minimum pH is 8 to 10, and the preferable range is pH 10 to 11. A range of the temperature at which the aqueous solution is kept is that wherein the maximum is 37 to 50° C., and the minimum is 0 to 4° C., the preferable range is 4 to 37° C., and the more preferable range is 4 to 10° C.

The complex of the OCIF substance and the polysaccharide substance is a complex wherein the OCIF substance and the polysaccharide substance are bound each other by a chemical bonding such as a covalent bond, an ionic bond, a coordinate bond and the like, or a non-chemical bond such as a hydrophobic interaction, a hydrogen bond, an electrostatic interaction, affinity binding and the like.

The complex does not contain a free polysaccharide substance.

A method of removing the free polysaccharide substance is not limited, as far as it is a method usually applied to a procedure such as purification, isolation, and fractionation. Examples of such methods may include: an ion exchange chromatography, an adsorption chromatography, partition chromatography, gel-filtration (molecular sieving) chromatography, hydrophobic chromatography, affinity chromatography, crystallization, salting out, an ultrafiltration, and the like. A gel filtration chromatography (hereinafter referred to as “gel filtration”), an ultrafiltration or the like is preferable.

Moreover, the complex of the OCTF substance and the polysaccharide substance of the present invention can be distinguished from the OCIF substance as a starting material, by measurement of an isoelectric point, measurement of a sugar content, and a quantifying method using an antibody or the like.

An isoelectric point can be measured according to a conventional method by the isoelectric focusing. Although OCIF is a basic protein and the isoelectric point is near pH 9, an isoelectric point will fall when the polysaccharide like dextran sulfate or a derivative thereof is bound thereto. Accordingly, it is possible to distinguish the OCIF substance from the complex of them and the polysaccharide substance.

A sugar content can be suitably measured using a method for quantifying neutral sugar according to a phenol sulfuric acid method. Since the sugar content of the OCIF to which polysaccharide substance is bound increases as compared with the original OCIF, it is possible to distinguish OCIF substance, from the complex of them and the polysaccharide substance.

The OCIF substance and a complex of them and a polysaccharide substance can be distinguished, by quantifying a polysaccharide substance in each of them, using an antibody specifically binding to the polysaccharide substance of the present invention.

The OCIF substance can be measured by ELISA method using an anti-OCIF monoclonal antibody or the like. Examples of the anti-OCIF monoclonal antibody preferably used for the ELISA include: the antibody produced by hybridoma OI-19 (FERM BP-6420), the antibody produced by hybridoma OI-4 (FERM BP-6419), the antibody produced by hybridoma OI-26 (FERM BP-6421) (see WO99/15691). The antibody produced by hybridoma OI-19 or OI-4 is bound to an OCIF monomer (monomer) and dimer (homodimer), and the antibody produced by hybridoma OI-26 is specifically bound to OCIF dimer (homodimer). ELISA measurement can be performed using these antibodies according to a conventional method (see WO99/15691).

A retentivity in blood in a body of human or animals of the complex of the OCIF substances and the polysaccharide substance can be measured and calculated by administrating it to human or animals, taking blood thereof, measuring concentration of the complex in the blood or serum according to ELIZA using an anti-OCIF monoclonal antibody as described above. (see WO99/15691).

A pharmaceutical composition containing the complex of the OCIF substances and the polysaccharide substance of the present invention is useful for prevention or treatment of bone metabolic diseases. The pharmaceutical composition of the present invention can be used as an agent for prevention or treatment of bone metabolic diseases.

Bone metabolic disease in the present invention may include: primary osteoporosis (senile osteoporosis, postmenopausal osteoporosis and idiopathic juvenile osteoporosis), and endocrine osteoporosis (hyperthyroidism, hyperparathyroidism, Cushing's syndrome and acromegaly), and osteoporosis accompanying hypogonadism (hypopituitarism, a Klinefelter syndrome and a Turner syndrome), hereditary and congenital osteoporosis (osteogenesis imperfecta, homocystinuria, Menkes syndrome, and Riley-Day syndrome), osteopenia due to gravity load mitigation or fixation and immobilization of limbs, Paget's disease, osteomyelitis, infectiosity focus due to loss of bone, hypercalcemia resulting from solid carcinoma (breast carcinoma, lung cancer, kidney cancer, prostatic cancer or the like) a hemology-malignant disease (multiple myeloma, lymphoma and leukemia), idiopathic hypercalcemia, hypercalcemia accompanying hyperthyroidism or kidney malfunction, osteopenia resulting from steroid medication, and osteopenia resulting from administration of other medicines (immunosuppresants such as methotrexate and ciclosporin A, heparin and antiepileptic), osteopenia accompanying kidney malfunction, osteopenia accompanying a surgical operation and digestive organ disease (small intestine hindrance, large intestine hindrance, chronic hepatitis, gastrectomy, primary biliary liver cirrhosis, and liver cirrhosis), osteopenia by various rheumatism, such as rheumatoid arthritis, osteoclasis and joint destruction by various rheumatism such as rheumatoid arthritis, mucilance type rheumatism, osteoarthritis, loss of periodontal bone, cancer metastasis to bone (osteolysis metastasis), Osteonecrosis or osteocyte death accompanying traumatic injury, Gaucher's disease, sickle cell anemia, systematic erythematosus lupus or nontraumatic injury, osteodystrophy such as renal osteodystrophy, osteopenia accompanying hypophosphatasia, diabetes, osteopenia accompanying nutritional disorder or eating disorders, and other osteopenia.

Moreover, the pharmaceutical composition of the present invention contains one or more other components in addition to the complex of the OCIF substance and the polysaccharide substance. There can be mentioned as an example of such a component the substance suppressing production of prostaglandin (hereinafter referred to as “PD”) and/or the substance competing an action of prostaglandin (hereinafter referred to as “anti-PD substance”), or the like. The present invention also relates to the pharmaceutical composition containing one or more of components other than the complex of the OCIF substance and the polysaccharide substance. In the present invention, “combined use” of the complex of the OCIF substance and the polysaccharide substance and the above-mentioned other components means using such pharmaceutical composition for prevention or treatment of bone metabolic disease.

Similarly, an agent for treatment or prevention of bone metabolic diseases of the present invention contains one or more of other components as an active ingredient in addition to the complex of the OCIF substance and the polysaccharide substance. Examples of such a suitable component may include anti-PD substance or the like. The present invention also relates to the agent for treatment or prevention of bone metabolic diseases which contains the complex of an OCIF substance and a polysaccharide substance and one or more of other components as an active ingredient. In the present invention, “combined use” of the complex of the OCIF substance and the polysaccharide substance and the above-mentioned other components also means using such an agent for treatment or prevention of bone metabolic diseases for prevention or treatment of bone metabolic disease.

In the present invention, examples of the anti-PD substance include: non-steroidal anti-inflammatory drugs (hereinafter referred to as “NSAIDs”), steroids, PD receptor antagonist, PD repressor, anti-PD activity neutralizing antibody, anti-PD receptor activity neutralizing antibody, the substance suppressing production of an inflammatory cytokine which promotes or induces production of PD, the substance suppressing the action of the cytokine or competing the action, an anti-cytokine activity neutralizing antibody, a cytokine acceptor activity neutralizing antibody, or the like, preferably NSAIDs, steroid, and PD receptor antagonist, and more preferably NSAIDs. Examples of NSAIDs include: acidic non-steroidal drugs such as a cyclooxygenase repressor, basic non-steroidal drugs or the like. More specific examples of the acidic non-steroidal drugs include: loxoprofen sodium (Japanese Patent Publication No. 58-4699 and U.S. Pat. No. 4,161,538), indomethacin (U.S. Pat. No. 3,161,654), mefenamic acid (U.S. Pat. No. 3,138,636), ibuprofen (U.S. Pat. No. 3,385,886), diclofenac sodium (U.S. Pat. No. 3,558,690), naproxen (U.S. Pat. No. 4,009,197), feprazone (U.S. Pat. No. 3,703,528), pranoprofen (U.S. Pat. No. 3,931,205), fentiazac (U.S. Pat. No. 3,476,766), sulindac (U.S. Pat. No. 3,654,349), clidanac (U.S. Pat. No. 3,565,943), aspirin DL-lysine (FR2115060), diflunisal (U.S. Pat. No. 3,714,226), fenoprofen calcium (U.S. Pat. No. 3,600,437), tiaprofenic acid (DE2055264 and GB-A-1331505), tolfenamic acid (U.S. Pat. No. 3,313,848), acemetacin (U.S. Pat. No. 3,910,952), piroxicam (U.S. Pat. No. 3,591,584), emorfazone (JP 72-24030 and U.S. Pat. No. 5,583,222), floctafenine (U.S. Pat. No. 3,644,368), tenoxicam (DE2537070 and GB-A-1519811), amfenac sodium (U.S. Pat. No. 4,045,576), oxaprozen (U.S. Pat. No. 3,578,671), alminoprofen (U.S. Pat. No. 3,957,850), celecoxib (WO95/15316 and U.S. Pat. No. 5,521,207 and Thomas D, P., et al., J. Med. Chem., 40. 1347-1365 (1997)) which is an inhibitor selective for cyclooxygenase-2, valdecoxib (U.S. Pat. No. 5,859,257), meloxicam (U.S. Pat. No. 4,233,299), tiracoxib (U.S. Pat. No. 5,994,381), etoricoxib, rofecoxib (WO95/00501 and U.S. Pat. No. 5,474,995), or the like. More specific examples of the basic non-steroidal drugs inculde: mepirizole (ZA6704936), tiaramide hydrochloride (U.S. Pat. No. 3,661,921) or the like. Examples of the steroids include: prednisolone (U.S. Pat. No. 2,837,464), dexamethasone (U.S. Pat. No. 3,007,923), betamethasone (U.S. Pat. No. 3,053,865), halopredone acetate (U.S. Pat. No. 4,226,862), or the like (Shimizu et al., Shikkantochiryou, the 3rd edition of revision, published by Nankoudou (1992). As the PD receptor antagonist, ONO-8711 (Watanabe, K., et al., Cancer Res., 59,.p5093 (1999)), ONO-8713 (Watanabe, K., et al, Cancer Lett, 156, p57 (2000)), or the like can be illustrated. The most preferred anti-PD substances for the present invention are loxoprofen sodium and celecoxib among them.

The above-mentioned pharmaceutical compositions provided by the present invention may be orally or parenterally administrated to human or animals other than human safely. Dosage form as a pharmaceutical composition may be suitably selected depending on the kind of diseases, the level of diseases, the age, the sex, the weight or the like. For example, it may be administrated orally in the form of tablets, capsules, powders, granules or syrups; injected in the form of an injection, intravenously alone or with conventional adjuncts such as glucose, amino acids or the like, or intramuscularly, subcutaneously, intracutaneously or intraperitoneally alone; administrated transdermally in the form of cataplasma; administrated transnasally in the form of a nasal drop; administrated transmucosaly or to the oral cavity in the form of a mucous membrane applying agent; or administrated intrarectally in the form of suppository. These preparations can be formulated with well-known auxiliary agent generally used in the field of medicine, such as excipients, binding agents, disintegrants, lubricants, flavoring agents, solubilizers, suspending agents, colorants, pH regulator, antiseptics, a gelling agent, surfactants a coating agent or the like according to a conventional method.

In the composition (also referred to as “preparation”) in the form of a tablet, any carriers known in the art can be used. The carriers include, for example, excipients such as lactose, white sugar, sodium chloride, glucose, urine, starch, calcium carbonate, kaolin, crystalline cellulose, silicate or the like; binding agents such as water, ethanol, propanol, simple syrup, glucose solution, starch solution, gelatin solution, carboxymethyl cellulose, shellac, methyl cellulose, potassium phosphate, polyvinyl pyrrolidone or the like; disintegrants such as dry starch, sodium alginate, agar powder, laminaran powder, sodium hydrogen carbonate, calcium carbonate, polyoxyethylene sorbitan fatty acid esters, sodium lauryl sulfate, stearic acid monoglyceride, starch, lactose or the like; decomposition inhibitors such as white sugar, stearin, cacao butter, hydrogenated oil or the like; absorption accelerators such as quaternary ammonium base, sodium lauryl sulfate or the like; moisturizers such as glycerin, starch or the like; adsorbents such as starch, lactose, kaolin, bentnite, colloidal silicate or the like; lubricants such as refined talc, stearate, boric acid powder, polyethylene glycol or the like. In addition, the tablet may be a tablet applied with general coatings, for example, sugar coating tablet, gelatin coating tablet, enteric coating tablet, film coating tablet, two layered tablet, multi-layered tablet or the like, if needed.

In the composition in the form of a pill, the composition may contain carriers known in the art, for example, excipients such as glucose, lactose, cacao butter, starch powder, hardened vegetable oil, kaolin, talc or the like; binding agents such as gum arabic powder, tragacanth powder, gelatin, ethanol or the like; disintegrants such as laminaran, agar or the like.

In the composition in the form of a suppository, the composition may contain carriers such as polyethylene glycol, cacao butter, higher alcohol, esters of higher alcohol, gelatin, semi-synthesized glyceride or the like.

In the composition in the form of an injection, it is preferable that the composition in the form of a solution or suspension is sterilized and is made isotonic with blood. When the composition is formed in the form of a solution, an emulsion or a suspension, a variety of dilutions known in the art can be used, including, for example, water, ethanol, propylene glycol, ethoxylated isostearyl alcohol, polyoxylated isostearyl alcohol, polyoxyethylene sorbitan fatty acid esters or the like. In addition, in this case, the composition may contain salts, glucose, glycerin or the like in an amount enough to maintain the isotonicity with blood, and a solubilizer, a buffering agent, a soothing agent, a pH regulator, a stabilizer, a solubilizing agent or the like. An injection can be freeze-dried.

Moreover, if needed, the composition may contain a coloring agent, a preservative, a perfume, a flavoring agent, a sweetener or other medicines, or the like.

The amount of the complex of the OCIF substance and the polysaccharide substance in the above-mentioned pharmaceutical compositions is not specifically limited, but are usually 0.1 to 70% by weight, and preferably 1 to 30% by weight.

The dose of the complex of the OCIF substance and the polysaccharide substance when it is administrated solely without being used together with the anti-PD agent depends on the condition, the age, the body weight, the form of administration or the dosage form or the like, and is generally, per a day for an adult, in the range wherein the upper limit is 30 to 1000 mg and the lower limit is 0.001 to 0.03 mg, and the preferable range is 0.03 to 30 mg.

The dose of the complex of the OCIF substance and the polysaccharide substance provided by the present invention in the case of the combined use with the anti-PD agent depends on the condition, the age, the body weight, the form of administration or the dosage form or the like, and is generally, per a day for an adult, in the range wherein the upper limit is 1 to 50 mg/kg and the lower limit is 0.01 to 0.1 mg/kg, and the preferable range is 0.01 to 1 mg/kg, and the more preferable range is 0.1 to 1 mg/kg.

The dose of the anti-PD agent in the case of the combined use of the complex of the OCIF substance and the polysaccharide substance and the anti-PD agent depends on the kind of the anti-PD agent, the kind of the complex of the OCIF substance and the polysaccharide substance used together and the dose thereof, the condition, the age, the body weight, the form of administration or the dosage form or the like, and is generally, per a day for an adult, in the range wherein the upper limit is 1 to 10 mg/kg and the lower limit is 0.01 to 0.1 mg/kg, and the preferable range is 0.01 to 1 mg/kg, and the more preferable range is 0.1 to 1 mg/kg. The dose of loxoprofen in the case of the combined use of the complex of the OCIF substance and the polysaccharide substance and loxoprofen as the preferred anti-PD agent is generally, per a day for an adult, in the range wherein the upper limit is 1 to 10 mg/kg and the lower limit is 0.01 to 0.1 mg/kg, and the preferable range is 0.01 to 1 mg/kg. Similarly, the dose of celecoxib in the case of the combined use of the complex of the OCIF substance and the polysaccharide substance and celecoxib as the preferred anti-PD agent is generally, per a day for an adult, in the range wherein the upper limit is 5 to 50 mg/kg and the lower limit is 0.1 to 1 mg/kg, and the preferable range is 1 to 5 mg/kg.

In the case of the combined use of the complex of an OCIF substance and a polysaccharide substance and anti-PD substance, the anti-PD substance may be administrated to the patient before, at the same time of or after administrating the complex of an OCIF substance and a polysaccharide substance. It can be administrated in a similar method to the administration method of the complex of an OCIF substance and a polysaccharide substance, but it is not limited thereto.

The pharmaceutical composition provided for the combined use of the complex of an OCIF substance and a polysaccharide substance and anti-PD substance is included in the pharmaceutical composition of the present invention, irrespective of the time of administrating the complex of an OCIF substance and a polysaccharide substance, the time of administrating the anti-PD substance, the administration method of both of them or the like. Therefore, the pharmaceutical composition provided for the combined use according to the present invention can be in any well-known form which is conventionally used for administrating plural different components to the same individual in the art, as in the case of a compounding agent, a combined use agent, or the like.

Furthermore, an agent for treatment or prevention of bone metabolic diseases provided for the combined use of the complex of an OCIF substance and a polysaccharide substance and anti-PD substance is included in the agent for treatment or prevention of bone metabolic diseases of the present invention, irrespective of the time of administrating the complex of an OCIF substance and a polysaccharide substance, the time of administrating the anti-PD substance, the administration method of both of them or the like. Therefore, the agent for treatment or prevention of bone metabolic diseases provided for the combined use according to the present invention can be in any well-known form which are conventionally used for administrating plural different components to the same individual in the art, as in the case of a compounding agent, a combined use agent, or the like.

The pharmaceutical composition provided by the present invention is administrated one time per several days, one time per a day or several times per a day depending on the kind of the active ingredient contained in the pharmaceutical composition, the form of administration, the dosage form or the like. The agent for treatment or prevention of bone metabolic diseases is administrated one time per several days, one time per a day or several times per a day depending on the kind of the active ingredient contained in the pharmaceutical composition, the form of administration, the dosage form or the like.

### EXAMPLES

The present invention is explained in detail with the following Examples and Preparation examples, but the present invention is not limited thereto.

### Preparation Example 1

### Preparation of a Complex of OCIF and Dextran Sulfate (I)

A recombinant human OCIF (a dimer) with a molecular weight of about 120000 obtained by a method described in WO96/26217 was dissolved in 10 mM sodium phosphate buffer solution (pH 10.5) containing 0.15 M sodium chloride at a concentration of 2, 5, 6.5, 10, 12.5, 20 or 50 mg/ml. Dextran sodium sulfate sulfur 5 (manufactured by Meito Industry, hereinafter referred to as “DS5”) was dissolved in the aqueous solution so that the final concentration might be 40, 100, 130, 150, 200, 400 and 510 or 1000 mg/ml, and then sodium hydroxide was added thereto to achieve pH 10.5 or 11. The obtained aqueous solution was kept at a temperature of 4, 7, or 37° C. for 1, 3, 6, 18, 24, 48, 72, 144, or 288 hours.

4 ml of the aqueous solutions after keeping a temperature was added to a gel-filtration column Superdex 200 pg (inside diameter of the column: 16 mm, length: 60 cm, exclusion-limit molecular weight: 1,300,000, manufactured by Amersham Pharmacia Biotech) which was equilibrated in advance with 10 mM sodium-phosphate buffer solution (pH 6) containing 0.3 M sodium chloride, and eluted at a flow rate of 2 ml/min with the buffer solution. Absorption at λ 280 nm was monitored using a ultraviolet spectrophotometer, and the eluate at a retention time of about 28 to 36 minutes was fractionated. Free DS5 which was not bound to the OCIF dimer was eluted at a retention time of about 50 to 70 minutes. The above-mentioned gel filtration was performed at a room temperature.

The obtained fraction containing the complex of OCIF and DS5 was freezed and stored at −60° C.

The reaction condition of each complex described in this Preparation Example was summarized in the following Table 1.

TABLE 1

| | DS5 | OCIF | | | Time of |
|---|---|---|---|---|---|
| | Concen- | Concen- | Temper- | | Keeping |
| | tration | tration | ature | | temperature |
| Preparation | (mg/ml) | (mg/ml) | (° C.) | pH | (hours) |
| Preparation 1 | 130 | 6.5 | 4 | 10.5 | 18 |
| Preparation 2 | 510 | 6.5 | 4 | 10.5 | 18 |
| Preparation 3 | 130 | 6.5 | 4 | 11 | 18 |
| Preparation 4 | 100 | 5 | 4 | 10.5 | 18 |
| Preparation 5 | 100 | 5 | 4 | 10.5 | 72 |
| Preparation 6 | 100 | 5 | 4 | 10.5 | 48 |
| Preparation 7 | 100 | 5 | 4 | 10.5 | 144 |
| Preparation 8 | 100 | 5 | 4 | 10.5 | 288 |
| Preparation 9 | 400 | 20 | 4 | 10.5 | 18 |
| Preparation 10 | 200 | 10 | 4 | 10.5 | 18 |
| Preparation 11 | 100 | 5 | 4 | 10.5 | 18 |
| Preparation 12 | 40 | 2 | 4 | 10.5 | 18 |
| Preparation 13 | 1000 | 12.5 | 4 | 10.5 | 18 |
| Preparation 14 | 1000 | 50 | 4 | 10.5 | 18 |
| Preparation 15 | 1000 | 12.5 | 4 | 10.5 | 18 |
| Preparation 16 | 1000 | 5 | 4 | 10.5 | 18 |
| Preparation 17 | 1000 | 2 | 4 | 10.5 | 18 |
| Preparation 18 | 150 | 5 | 37 | 10.5 | 1 |
| Preparation 19 | 150 | 5 | 37 | 10.5 | 3 |
| Preparation 20 | 150 | 5 | 37 | 10.5 | 6 |
| Preparation 21 | 150 | 5 | 37 | 10.5 | 24 |
| Preparation 22 | 150 | 5 | 7 | 10.5 | 168 |

### Preparation Example 2

### Preparation of a Complex of OCIF and Dextran Sulfate (II)

The human OCIF (dimer) described in Preparation Example 1 was dissolved in 10 mM sodium phosphate buffer solution (pH 10.5) containing 0.15 M sodium chloride at a concentration of 5 mg/ml. Dextran sodium sulfate with a molecular weight 5000 (manufactured by Wako Pure Chemical Industries, Ltd., hereinafter referred to as “DS5000”) was dissolved in the aqueous solution so that the final concentration might be 150 mg/ml, and then sodium hydroxide was added thereto to achieve pH 10.5. The obtained aqueous solution was kept at a temperature of 4° C. for 24 hours.

4 ml of the aqueous solutions after keeping a temperature was added to a gel-filtration column Superdex 200 pg (inside diameter of the column: 16 mm, length: 60 cm, exclusion-limit molecular weight: 1,300,000, manufactured by Amersham Pharmacia Biotech) which was equilibrated in advance with 10 mM sodium-phosphate buffer solution (pH 6) containing 0.3 M sodium chloride, and eluted at a flow rate of 2 ml/min with the buffer solution. Absorption at λ 280 nm was monitored using a ultraviolet spectrophotometer, and the eluate at a retention time of about 28 to 36 minutes was fractionated. Free DS5000 which was not bound to the OCIF dimer was eluted at a retention time of about 40 to 65 minutes. The above-mentioned gel filtration was performed at a room temperature.

The obtained fraction containing the complex of OCIF and DS5 was freezed and stored at −60° C.

The reaction condition of each complex indicated by this Preparation example was summarized in the following Table 2.

TABLE 2

| | DS5000 | OCIF | | | Time of |
|---|---|---|---|---|---|
| | Concen- | Concen- | | | Keeping |
| | tration | Tration | Temperature | | temperature |
| Preparation | (mg/ml) | (mg/ml) | (° C.) | pH | (hours) |
| Preparation 23 | 150 | 5 | 4 | 10.5 | 24 |

In the following examples, the prepared pharmaceutical composition is referred to as “Preparation”.

### Preparation Example 3

### Measurement of an Isoelectric Point

The recombinant human OCIF (a dimer) described in Preparation Example 1 and the complex of OCIF and DS5 obtained in Example 1 (Preparation 22) were added to isoelectric focusing gel IEF PAGE mini (pH range of 3 to 10, manufactured by Iwaki Glass), and predetermined voltage was applied according to description of the description of the equipment. The gel was stained with Coomassie Blue after electrophoresis.

As a result, the isoelectric point of OCIF was about pI9, and the isoelectric point of the complex of OCIF and DS5 (Preparation 22) was about pI6.5.

### Preparation Example 4

### Quantification of OCIF and Dextran Sulfate in the Complex of OCIF and Dextran Sulfate, and Calculation of a Molecular Ratio Thereof

1) Preparation of a Stock Solution of an Anti-Human OCIF Monoclonal Antibody OI-4 Labeled with Peroxidase

The antibody was labeled with enzyme using EZ-Link Maleimide Activated Horseradish Peroxidase Kit (manufactured by Pierce) according to the protocol II of the kit.

To one ml of a solution of the purified anti-human OCIF antibody (see WO99/15691) OI-4 diluted with 10 mM phosphoric acid buffer solution (pH 7.6) so that the concentration may be 1 mg/ml, was added 4 μl of N-sccinimidyl-S-acetyl thioacetic acid appended to the above-mentioned kit dissolved with dimethyl formamide so that the concentration might be 10 mg/ml just before use, and it was kept at the room temperature for 30 minutes. After adding 20 μl of solutions wherein 5 mg hydroxylamine hydrochloride was dissolved in 100 μl of Maleimide Conjugation Buffer appended to the above-mentioned kit in advance just before use thereto, it was kept at the room temperature for 2 hours. The resultant reaction mixture was added to a polyacrylamide desalting column (a volume of 10 ml) equilibrated in advance with 30 ml of Maleimide Conjugation Buffer appended to the above-mentioned kit, and was eluted with Maleimide Conjugation Buffer, and fractionated in 0.5 ml each. 7th to 10th fractions containing the antibody were combined. Thereto was added 100 μl of the solution wherein 5 mg of EZ-Link Maleimide Activated Horseradish Peroxidase appended to the above-mentioned kit was dissolved in 500 μl of distilled water, kept at a room temperature for one hour, and then equal amount of glycerol was added thereto, and stored at −20° C.

The finally obtained solution was used as a stock solution of anti-human OCIF monoclonal antibody OI-4 labeled with peroxidase (hereinafter referred to as POD-OI-4″)

2) Quantification of OCIF

The amount of OCIF in each complex given in Preparation Examples 1 and 2 was measured by ELISA method using an anti-OCIF monoclonal antibody.

To each well of a 96 well micro titer plate (Maxisorp, manufactured by NUNC) was poured 100 μl each of the solution wherein anti-human OCIF monoclonal antibody OI-26 (see WO99/15691) was dissolved in 0.1 M NaHCO₃ solution so that the concentration might be 5 μg/ml, sealed, and allowed to stand at 4° C. overnight.

Each well was washed three times with 250 μl of PBS (pH 7.4) containing 0.1% Polysorbate 20.

To each well was added 20 μl of a dilution buffer solution (composition: 0.2 M Tris-hydrochloric acid and 40% Block Ace (manufactured by Snow Brand Milk Products Co., Ltd.), 0.1% Polysorbate 20: pH 7.4), and it was kept at the room temperature for 20 minutes.

The samples to be tested were suitably diluted using the dilution buffer solution. In order to make a calibration curve, the dilution buffer solution containing OCIF at known concentration was used as a standard solution, and the dilution buffer solution was used as a control. 50 μl of each sample to be tested was poured into each well.

To each well was added 50 μl of the solution wherein the stock solution of POD-OI-4 obtained in 1) was diluted with [0.1 M Tris-hydrochloric acid, 40% Block Ace (manufactured by Snow Brand Milk Products Co., Ltd.), and 0.1% polysorbate 20 (pH 7.4)] 1500 times, and allowed to stand at the room temperature for 2 hours.

After the reaction, each well was washed 4 times with 250 μl of phosphate buffered saline containing a 0.1% polysorbate 20 (hereinafter referred to as “PBS”, pH 7.4).

0.1 M citric acid and 0.2 M disodium hydrogenphosphate were mixed, and used as a substrate solution (pH 4.5).

To each well was added 100 μl of a substrate liquid obtained by adding 32.5 ml of the substrate solution and 6.5 μl of hydrogen peroxide to 13 mg of OPD tablet (manufactured by Wako Pure Chemical Industries, Ltd.) to dissolve it, and it was shaded with aluminum foil, and allowed to stand at the room temperature for 15 minutes.

To each well was added 50 μl of a reaction stopping solution obtained by mixing 250 ml of purified waters and 50 ml of sulfuric acid.

After stirring quietly using a churning shaking machine (titer mixer MB-1: manufactured by Japan Trika), the absorbance of the sample solution at a wavelength of λ490 nm was measured using a micro plate leader (SPECTRA FLUOR: manufactured by TECAN).

From the calibration curve created using the standard solution, OCIF concentration in a sample to be tested was calculated.

3) Quantification of Dextran Sulfate

An amount of dextran sulfate in each complex described in Examples 1 and 2 was measured as neutral sugar by a phenol sulfuric acid method.

A known concentration in the range of 10 to 60 μg/ml of DS5 (manufactured by Meito Industry) or DS5000 (manufactured by Wako Pure Chemical Industries, Ltd.) was dissolved in the diluting solution (composition: 0.01 M citric acid, 0.3 M sodium chloride, 0.01% polysorbate 80 aqueous solution: pH 6.0), and used as a standard solution. 0.2 ml each of the standard solution, a sample solution, and diluting solution were poured into each of test tubes.

0.2 ml of 50 mg/ml phenol aqueous solution was poured into each of the test tubes, and stirred promptly.

After keeping the solution at 60° C. for 20 seconds in a water bath, 1.0 ml of concentrated sulfuric acids was added thereto, and stirred promptly.

It was allowed to stand for 10 minutes at a room temperature, then stirred again, and allowed to stand for 20 minutes at the room temperature.

Absorbance of each well at wavelength X490 nm was measured using a spectrophotometer (UV-240: manufactured by Shimadzu Seisakusho, K. K.).

A sugar chain is bound to human OCIF itself. Therefore, an amount of the dextran sulfate bound to OCIF was calculated by deducting the value of the neutral sugar content of OCIF as a raw material measured in the same way as above from the value of the neutral sugar content in the complex of OCIF and dextran sulfate obtained by the above-mentioned measurement.

4) Calculation of a Molecular Ratio of OCIF and Dextran Sulfate in the Complex of OCIF and Dextran Sulfate

The amount of dextran sulfate in each complex measured by the above 3) was divided by the amount of OCIF in each complex measured in the above 2), to calculate an amount of the dextran sulfate bound to 1 mg of the OCIF.

Subsequently, a molecular ratio of the OCIF monomer and the dextran sulfate in each complex was calculated as a number of molecular of the dextran sulfate per one molecule of OCIF monomer, regarding a molecular weight of human OCIF monomer as 60000, a molecular weight of DS5 as 1950, a molecular weight of DS5000 as 5000, a molecular weight of DS10000 as 10000.

Results are shown in the following table 3.

TABLE 3

| | | Molecular ratio of |
|---|---|---|
| | Amount of the dextran | OCIF monomer and |
| | sulfate in a complex | dextran sulfate in |
| Preparation | (μg/mgOCIF) | complex |
| Preparation 1 | 48.7 | 1:1.5 |
| Preparation 2 | 100.2 | 1:3.1 |
| Preparation 3 | 39.7 | 1:1.2 |
| Preparation 4 | 54.9 | 1:1.7 |
| Preparation 5 | 62.0 | 1:1.9 |
| Preparation 6 | 60.7 | 1:1.9 |
| Preparation 7 | 58.5 | 1:1.8 |
| Preparation 8 | 60.3 | 1:1.9 |
| Preparation 9 | 67.7 | 1:2.1 |
| Preparation 10 | 94.3 | 1:2.9 |
| Preparation 11 | 63.6 | 1:2.0 |
| Preparation 12 | 60.8 | 1:1.9 |
| Preparation 13 | 144.9 | 1:4.5 |
| Preparation 14 | 116.4 | 1:3.6 |
| Preparation 15 | 185.2 | 1:5.7 |
| Preparation 16 | 145.0 | 1:4.5 |
| Preparation 17 | 116.5 | 1:3.6 |
| Preparation 18 | 46.0 | 1:1.4 |
| Preparation 19 | 61.0 | 1:1.9 |
| Preparation 20 | 68.3 | 1:2.1 |
| Preparation 21 | 110.7 | 1:3.4 |
| Preparation 22 | 100.3 | 1:3.1 |
| Preparation 23 | 243.9 | 1:2.9 |

### Example 1

### Combined use of the Complex of OCIF and Dextran Sulfate and Anti-PD Agent (1)

This example was carried out according to a method of Winter et al. (C. V. Winder, et al. Arthritis Rheum 12 and p472 (1957)).

Thermally killed cells of Mycobacterium butyricum were pulverized in an agate mortar, suspended in a liquid paraffin sterilized with a hot air so that the concentration thereof might be 2 mg/ml and subsequently subjected to ultrasonication. Thereby, an adjuvant was produced.

50 μl each of the obtained adjuvant was injected to two parts in a skin of the tail root part of Lewis rat (eight-week old, female) (200 μg in total per one rat), to induce arthritis in this rat.

From the 14th day to the 20th after injection of the adjuvant, loxoprofen sodium (Japanese Patent Publication No. 58-4699) suspended in a 0.5% Tragacanthaqueous solution (manufactured by Nihon Funmatsu Yakuhin Co.,Ltd.) so that the concentration thereof may be 1 mg/ml was orally administrated to the rat at a rate of 2 ml/kg every day so that the dose per weight might be 5 mg/kg. Preparation 4 described in Preparation Example 4 which was previously dissolved in a physiological saline (manufactured by Otsuka Phamaceutical Co.,Ltd.) containing 0.1 mg/mg of Tween 80 (manufactured by Sigma) was administrated intraperitoneally everyday at a rate of 2 ml/kg to the same rat so that the dose per weight might be 0.1 mg/kg. As a control, instead of at least one of loxoprofen sodium and Preparation 4, only the solvent used for dissolving them was administrated to the rat.

The rat was dissected at the 21st day after injection of the adjuvant, and the right-and-left thighbone was extracted. The bone density of the distal end of the right-and-left thighbone was measured using the X ray measurement apparatus (Dual energy X-ray absorptiometry DCS-600R, manufactured by Aroka), and the average of each of the measured values was calculated, and was defined as a bone density of the individual.

As for the difference between the averages of the bone density of each group, a significant difference was tested by Student's t-test, and the P value was calculated again.

The result is shown in Table 4.

TABLE 4

| | Bone density (mg/cm²) |
|---|---|
| Administrated Preparation | (average ± standard deviation) |
| Solvent | 106.4 ± 1.0 |
| Loxoprofen sodium | 106.5 ± 0.8 |
| Preparation 4 | 109.6 ± 2.1 |
| Loxoprofen sodium and | 115.2 ± 2.1 |
| Preparation 4 | |

1) The P value concerning the difference of the bone density of a group to which a solvent was administrated and that of a group to which loxoprofen sodium and Preparation 4 was administrated was less than 0.01.

2) The P value concerning the difference of the bone density of a group to which loxoprofen was administrated and that of a group to which loxoprofen sodium and Preparation 4 were administrated was less than 0.01.

As shown in Table 4, by the combined use of loxoprofen sodium and preparation 4, there can be achieved the adjuvant-arthritis improvement effect which was significantly excellent, as compared with sole administration of the solvent or loxoprofen.

### Example 2

### Combined Use of the Complex of OCIF and Dextran Sulfate and the Anti-PD Agent (2)

Arthritis was induced in Lewis rat (eight-week old, female) according to the same method as Example 1.

From the 14th day to the 20th after injection of the adjuvant, the celecoxib (Thomas D, P., et al., J. Med. Chem., 40, and 1347-1365 (1997): WO95/15316) suspended in a 0.5% Tragacanthaqueous solution (manufactured by Nihon Funmatsu Yakuhin Co.Ltd. so that the concentration thereof might be 0.2 mg/ml was orally administrated to the rat at a rate of 2 ml/kg every day so that the dose per weight might be 1 mg/kg. Preparation 22 described in Preparation Example 1 which was previously dissolved in a physiological saline (manufactured bay Otsuka Pharmaceutical Co., Ltd.) containing 0.1 mg/ml % of Tween 80 (manufactured by Sigma) was administrated intraperitoneally only a day to the same rat so that the dose per weight might be 3.5 mg/kg at a rate of 2 ml/kg. As a control, instead of at least one of loxoprofen sodium and Preparation 22, only the solvent used for dissolving them was administrated to the rat.

The rat was dissected at the 21st day after injection of the adjuvant, and the right-and-left thighbone was extracted. The bone density of the distal end of the right-and-left thighbone was measured using the X ray measurement apparatus (Dual energy X-ray absorptiometry DCS-600R, manufactured by Aroka), and the average of each of the measured values was calculated, which was defined as a bone density of the individual.

The result is shown in Table 5.

TABLE 5

| | Bone density (mg/cm²) |
|---|---|
| Administrated Preparation | (average ± standard deviation) |
| Solvent | 103.3 ± 1.1 |
| Celecoxib | 103.0 ± 1.8 |
| Preparation 22 | 104.2 ± 2.1 |
| Celecoxib and Preparation 22 | 117.2 ± 1.9 |

1) The P value concerning the difference of the bone density of a group to which a solvent was administrated and that of a group to which celecoxib and Preparation 22 was administrated was less than 0.01.

2) The P value concerning the difference of the bone density of a group to which celecoxib was solely administrated and that of a group to which celecoxib and Preparation 22 were administrated was less than 0.001.

3) The P value concerning the difference of the bone density of a group to which Preparation 22 was solely administrated and that of a group to which celecoxib and Preparation 22 were administrated was less than 0.001.

As shown in Table 5, by the combined use of celecoxib and Preparation 22, there can be achieved the adjuvant-arthritis improving effect which was significantly excellent, as compared with sole administration of the solvent, celecoxib or Preparation 22.

The pharmaceutical composition containing the complex of the OCIF substance and a polysaccharide substance, as well as a substance suppressing production of prostaglandin and/or a substance competing an action of prostaglandin, provided by the present invention, has an outstanding retentivity in blood, and is useful for prevention and/or treatment of various bone metabolic diseases.

We claim:

1. A pharmaceutical composition which contains (i) a complex of (a) one or more OCIF substances selected from the group consisting of an osteoclastogenesis inhibitory factor, an analog thereof and a variant thereof, and (b) a polysaccharide substance comprising a polysaccharide or a derivative thereof, wherein the OCIF substance and the polysaccharide substance are bound in a molecular ratio of 1:1 to 1:8; and (ii) a substance suppressing production of prostaglandin and/or a substance competing an action of prostaglandin.

2. The pharmaceutical composition according to claim 1 wherein the polysaccharide or a derivative thereof is dextran sulfate.

3. The pharmaceutical composition according to claim 2 wherein an average molecular weight of the dextran sulfate is 1800 to 6000.

4. The pharmaceutical composition according to any one of claims 1 to 3 wherein the substance suppressing production of prostaglandin and/or the substance competing an action of prostaglandin is non-steroidal anti-inflammatory drug.

5. The pharmaceutical composition according to claim 4 wherein the non-steroidal anti-inflammatory drug is a cyclooxygenase inhibitor.

6. The pharmaceutical composition according to claim 5 wherein the cyclooxygenase inhibitor is loxoprofen sodium or celecoxib.

7. The pharmaceutical composition according to any one of claims 1 to 6 wherein the complex of the OCIF substance and the polysaccharide substance is produced by the following steps (i) and (ii):
(i) a step of mixing the OCIF substance and the polysaccharide substance; and
(ii) a step of removing a free polysaccharide.

8. The pharmaceutical composition according to claim 7 wherein the OCIF substance and the polysaccharide substance is maintained under alkaline condition in the step (i).

9. The pharmaceutical composition according to claim 8 wherein the OCIF substance and the polysaccharide substance is maintained at pH 10 to 11 in said step (i).

10. The pharmaceutical composition according to any one of claims 7 to 9 wherein the free polysaccharide is removed by gel filtration.

11. A method for treatment or prevention of a bone metabolic disease which comprises administering to a patient in need thereof, an effective amount of
(i) a complex of (a) one or more OCIF substances selected from the group consisting of an osteoclastogenesis inhibitory factor, an analog thereof and a variant thereof, and (b) a polysaccharide substance comprising a polysaccharide or a derivative thereof, wherein the OCIF substance and the polysaccharide substance are bound in a molecular ratio or 1:1 to 1:8; and of
(ii) a substance suppressing production of prostaglandin and/or a substance competing an action of prostaglandin.

12. The method according to claim 11 wherein the polysaccharide or a derivative thereof is dextran sulfate.

13. The method according to claim 12 wherein an average molecular weight of the dextran sulfate is 1800 to 6000.

14. The method according to any one of claims 11 to 13 wherein the substance suppressing production of prostaglandin and/or the substance competing an action of prostaglandin is a non-steroidal anti-inflammatory drug.

15. The method according to claim 14 wherein the non-steroidal anti-inflammatory drug is cyclooxygenase inhibitor.

16. The method according to claim 15 wherein the cyclooxygenase inhibitor is loxoprofen sodium.

17. The method according to claim 15 wherein the cyclooxygenase inhibitor is celecoxib.

18. The method according to any one of claims 11-17 wherein said complex is administered per day to a human in an amount in a range having an upper limit of 1 to 50 mg/kg and a lower limit of 0.01 to 0.1 mg/kg; and said substance suppressing production of prostaglandin and/or a substance competing an action of prostaglandin is administered per day to a human in an amount in a range having an upper limit of 1 to 10 mg/kg and a lower limit of 0.01 to 0.1 mg/kg.

19. The method according to claim 18 wherein said complex is administered in an amount of from 0.01 to 1 mg/kg and said substance suppressing production of prostaglandin and/or a substance competing an action of prostaglandin is administered in an amount of from 0.01 to 1 mg/kg.

20. The method according to claim 16 wherein said complex is administered per day to a human in an amount of from 0.01 to 1 mg/kg and wherein said loxoprofen sodium is administered per day to a human in an amount in a range wherein the upper limit is 1 to 10 mg/kg and the lower limit is 0.01 to 0.1 mg/kg.

21. The method according to claim 17 wherein said complex is administered per day to a human in an amount of from 0.01 to 1 mg/kg and wherein said celecoxib is administered per day to a human in an amount in a range wherein the upper limit is 5 to 10 mg/kg and the lower limit is 0.1 to 1 mg/kg.
